(19) United States
(12) Patent Application Publication
(10) Pub. No.: US 2020/0237875 A1
(43) Pub. Date: Jul. 30, 2020

(54) FORMULATIONS AND METHODS FOR TREATMENT OF FIBROMYALGIA AND RELATED MYOFASCIAL PAIN DISORDERS

(71) Applicant: SYNLEV PHARMACEUTICALS CORPORATION, Dover, DE (US)

(72) Inventor: Miguel Angel Pappolla, Houston, TX (US)

(73) Assignee: SYNLEV PHARMACEUTICALS CORPORATION, Dover, DE (US)

(21) Appl. No.: 16/777,274

(22) Filed: Jan. 30, 2020

Related U.S. Application Data
(60) Provisional application No. 62/798,828, filed on Jan. 30, 2019.

(51) Int. Cl. A61K 38/26; A61K 31/4965; A61K 31/18; A61K 31/4439; A61K 31/4453; A61K 31/403; A61K 31/4985; A61K 31/155; A61K 31/40; A61K 31/522; A61K 45/06; A61K 31/135; A61K 31/496; A61K 31/55; A61K 31/381; A61K 31/165; A61P 23/00

(52) U.S. Cl. CPC A61K 38/26; A61P 23/00; A61K 31/18; A61K 31/4439; A61K 31/4453; A61K 31/403; A61K 31/4985; A61K 31/155; A61K 31/40; A61K 31/522; A61K 45/06; A61K 31/135; A61K 31/496; A61K 31/55; A61K 31/381; A61K 31/165; A61K 31/4965

(57) ABSTRACT

Formulations and methods for treating fibromyalgia or chronic pain in humans comprising chronically administering to a human patient experiencing fibromyalgia who is experiencing insulin resistance a pharmaceutical composition comprising a therapeutically effective amount of a drug that treats insulin resistance and a therapeutically effective amount of a drug for treating the pain associated with fibromyalgia. In certain preferred embodiments, the formulation is an oral solid dosage form.

### FIELD OF THE INVENTION

The invention generally relates to formulations and methods for treatment of fibromyalgia and related myofascial pain disorders.

### BACKGROUND OF THE INVENTION

Fibromyalgia is one of the most frequent generalized pain disorders with poorly understood neurobiological mechanisms. Despite extensive research, the etiology of fibromyalgia is unknown, and thus, there is no disease-modifying therapy available for this condition. Afflicted patients have chronic widespread myofascial pain and protean somatic symptoms, including fatigue, nonrestorative sleep, gastrointestinal complaints, and problems of cognition and mood. Fibromyalgia is one of the most frequent generalized pain disorders with poorly understood neurobiological mechanisms. The global economic impact of FM is enormous. In the United States alone, the healthcare cost is around $100 billion/year; comparable to reports in European countries. Patients afflicted with FM have widespread chronic pain and protean somatic symptoms, including fatigue, nonrestorative sleep, gastrointestinal complaints, and problems of cognition and mood. Due to lower pain thresholds, patients with FM also have a higher incidence of symptomatic musculoskeletal and spinal disorders, which in themselves contribute to the financial burden of managing this disorder.

Fibromyalgia is a central sensitivity pain disorder characterized by abnormal processing of nociceptive stimuli. Many hypotheses were advanced to explain the extensive array of symptoms, including inherited abnormalities, dysfunction of neurotransmitters pathways such as substance, immune dysregulation, and several others. Unfortunately, none of these propositions has led to practical advances beyond symptomatic treatment. Because dysfunctions in the brain microvasculature are known to be caused by insulin resistance (leading to focal cerebral hypoperfusion), and because similar abnormalities are present in patients with fibromyalgia, it has been hypothesized that insulin resistance may be causally linked to fibromyalgia. In the process of seeking evidence in support of this hypothesis, several unexpected discoveries were made as described below; these constitute the bases for the claims in this application.

### OBJECTS AND SUMMARY OF THE INVENTION

It is an object of the invention to provide improved methods and formulations for treating fibromyalgia.

It is another object of the present invention to provide improved methods and formulations for treating certain types of chronic pain, e.g., chronic pain patients who are prediabetic or diabetic.

It is another object of the present invention to provide improved methods and formulations for treating myofascial pain, which is one of the main and most bothersome symptoms of fibromyalgia, but it may occur in isolation in patients not meeting established diagnostic criteria for fibromyalgia. In accordance with the above objects and others, the present invention is directed in part to a pharmaceutical formulation comprising (i) an effective amount of a drug for treating insulin resistance and (ii) an effective amount of a drug for fibromyalgia.

In accordance with the above objects and others, the invention is directed in part to a pharmaceutical composition comprising a therapeutically effective amount of a drug which treats insulin resistance, and a therapeutic amount of a drug for treating pain associated with fibromyalgia. In certain preferred embodiments, the drug for treating insulin resistance is selected from the group consisting of a biguanide, indole-3-propionic acid, a PPARγ agonist, a glucagon-like peptide-1 (GLP-1) agonist, a DPP-4 inhibitor, and combinations of any of the foregoing drugs. In other preferred embodiments, the drug for treating insulin resistance is selected from the group consisting of an indole-3-propionic acid, a PPARγ agonist, a glucagon-like peptide-1 (GLP-1) agonist, a DPP-4 inhibitor, and combinations of any of the foregoing drugs. In certain preferred embodiments, the drug for treating the pain associated with fibromyalgia is selected from the group consisting of a tricyclic antidepressant, a selective serotonin reuptake inhibitor, a selective norepinephrine reuptake inhibitor, an atypical antidepressant, a drug with membrane-stabilizing properties, and combinations of any of the foregoing drugs. The biguanide may be selected from the group consisting of glipizide, glyburide, pioglitazone, repaglinide, saxagliptin, sitagliptin, and metformin. The GLP-1 agonist may be selected from the group consisting of exenatide, liraglutide, lixisenatide, dulaglutide, and semaglutide. In other preferred embodiments, the drug which treats insulin resistance is a PPARγ agonist. The DDP-4 inhibitor selected from the group consisting of sitagliptin, vildagliptin, saxagliptin, and linagliptin. The tricyclic antidepressant may be selected from the group consisting of amitriptyline, desipramine, doxepin, imipramine, nortriptyline, amoxapine, clomipramine, maprotiline, trimipramine, and protriptyline. The atypical antidepressant selected from the group consisting of bupropion, trazodone, and mirtazapine. The drug for treating the pain associated with fibromyalgia is a selective norepinephrine reuptake inhibitor (SNRI) that may be selected from the group consisting of venlafaxine, desvenlafaxine, milnacipran, duloxetine, and levomilnacipran. The drug selective serotonin reuptake inhibitor (SSRI) selected from the group consisting of fluoxetine, sertraline, paroxetine, escitalopram, fluvoxamine, citalopram, vilazodone, and vortioxetine. The drug with membrane-stabilizing properties may be selected from gabapentin, pregabalin, carbamazepine, and oxcarbazepine. In certain preferred embodiments, the formulation is an oral formulation (liquid or solid). In certain preferred embodiments, the pharmaceutical composition is an oral solid dosage form, e.g., a tablet or a capsule. In further embodiments, the oral dosage form may be in immediate release, controlled (e.g., delayed) release or extended-release form. Immediate release, controlled release, and extended-release formulations may be given on a once a day basis, and such formulations are described in the following paragraphs. In certain preferred embodiments, the pharmaceutical composition comprises from about 25 to about 200 mg, preferably from about 25 mg to about 200 mg desipramine as the drug for treating pain associated with fibromyalgia. In certain embodiments, the pharmaceutical composition comprises from about 125 mg to about 200 mg desipramine. In other preferred embodiments, the pharmaceutical composition includes as the drug for treating pain associated with fibromyalgia from about 10 mg to about 375 mg, and preferably from about 37.5 mg to about 225 mg venlafaxine. In certain preferred embodiments, the drug for treating insulin resistance comprises or consists of from about 100 mg to about 2000 mg metformin, as a divided dose (e.g., administered twice a day) or as a single dose (e.g., administered once a day in extended-release form as described herein). In certain preferred embodiments, the drug which treats insulin resistance is a DPP-4 inhibitor such as sitagliptin in an amount from about 1 mg to about 500 mg, preferably from about 5 mg to about 300 mg per day, and as much as 500 mg per day). In certain embodiments, the pharmaceutical composition is an oral dosage form (tablet, capsule or liquid (solution, emulsion or suspension).

The invention is also directed to a method for treating fibromyalgia in humans, comprising chronically administering to a human patient experiencing fibromyalgia who is experiencing insulin resistance a therapeutically effective amount of a drug that treats insulin resistance and a therapeutically effective amount of a drug for treating the pain associated with fibromyalgia. The drug for treating insulin resistance is selected from the group consisting of a biguanide, indole-3-propionic acid, a PPARγ agonist, a glucagon-like peptide-1 (GLP-1) agonist, a DPP-4 inhibitor, and combinations of any of the foregoing and the drug for treating pain associated with fibromyalgia is selected from the group consisting of a tricyclic antidepressant, a selective serotonin reuptake inhibitor, a selective norepinephrine reuptake inhibitor, an atypical antidepressant, a drug with membrane-stabilizing properties, and combinations of any of the foregoing. Alternatively, the drug for treating insulin resistance is selected from the group consisting of indole-3-propionic acid, a PPARγ agonist, a glucagon-like peptide-1 (GLP-1) agonist, a DPP-4 inhibitor, and combinations of any of the foregoing drugs.

The invention is further directed in part to a method for treating chronic pain in humans, comprising long-term administration to a human patient experiencing chronic pain of central origin (e.g., myofascial pain and human patients affected by “fibromyalginess” as further defined below, or chronic pain associated with insulin resistance), a therapeutically effective amount of a drug which treats insulin resistance and a therapeutically effective amount of a drug for treating the pain associated with fibromyalgia. The drug for treating insulin resistance is selected from the group consisting of a biguanide, indole-3-propionic acid, a PPARγ agonist, a glucagon-like peptide-1 (GLP-1) agonist, a DPP-4 inhibitor, and combinations of any of the foregoing and the drug for treating pain associated with fibromyalgia is selected from the group consisting of a tricyclic antidepressant, a selective serotonin reuptake inhibitor, a selective norepinephrine reuptake inhibitor, an atypical antidepressant, a drug with membrane-stabilizing properties, and combinations of any of the foregoing. Alternatively, the drug for treating insulin resistance is selected from the group consisting of indole-3-propionic acid, a PPARγ agonist, a glucagon-like peptide-1 (GLP-1) agonist, a DPP-4 inhibitor, and combinations of any of the foregoing.

The invention is further directed in part to a method for treating fibromyalgia or myofascial pain in human patients, comprising determining if a human patient is prediabetic or diabetic, and if the patient is prediabetic or diabetic, chronically administering to the patient (i) an effective amount of a drug for treating insulin resistance and (ii) an effective amount of a drug for fibromyalgia.

The invention is also directed, in part, to a method for treating fibromyalgia or myofascial pain in patients without pre-diabetes or diabetes, by using (i) an effective amount of drug for treating insulin resistance and (ii), an effective amount of drug for fibromyalgia. The latter is supported by at least the following facts: (i) some of the drugs using to treat pre-diabetes have analgesic activity of their own and (ii), diagnostic tools to diagnose prediabetes can miss certain mechanisms that cause pain, which can also co-exist in pre-diabetic patients, but that may also occur in isolation with myofascial pain or fibromyalgia. Thus, it is hypothesized and believed that the pharmaceutical combinations embodied in the invention are also effective in such individuals (i.e., patients affected with myofascial pain or fibromyalgia without detectable pre-diabetes).

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to oral, and parenteral (e.g., intravenous) administration.

“Pharmaceutically acceptable” refers to those propetiies and/or substances that are acceptable to the patient from a pharmacological/toxicological point of view and to the manufacturing pharmaceutical chemist from a physical/chemical point of view regarding composition, formulation; stability, patient acceptance and bioavailability.

The term “treat” or “treating”, as used herein, means reducing the frequency with which symptoms are experienced by a subject or administering an agent or compound to reduce the frequency and/or severity with which symptoms are experienced.

As used herein, “alleviate” is used interchangeably with the term “treat.” Treating a disease, disorder or condition may or may not include complete eradication or elimination of the symptom.

The term “therapeutic” as used herein means a treatment and/or prophylaxis. Throughout this disclosure, various aspects of the invention can be presented in a range format.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

All illustrations of the drawings are for the purpose of describing selected versions of the present invention and are not intended to limit the scope of the present invention.

FIG. 1 is a diagram depicting the association between fibromyalgia and insulin resistance.

FIG. 2 is a diagram depicting the effect on pain scores of standard treatment (ST), metformin, and PPD-4 inhibitors, alone and in combination.

FIG. 2A is a diagram depicting the effect of metformin treatment on pain showing pain scores at presentation, after standard treatment, and after the addition of metformin.

FIG. 3 is a table diagram depicting class 1 and class 2 dosages used to treat fibromyalgia.

### DETAILED DESCRIPTION OF THE INVENTION

The present invention is directed in part to the surprising discovery that insulin resistance is a central metabolic abnormality in fibromyalgia (as well as other chronic pain disorders associated with centralized pain or “fibromyalginess” as further defined below) and by addressing it, in conjunction with the use of co-analgesic agents targeting central pain, chronic myofascial pain or associated symptoms, the pain present in any of these conditions can be treated more effectively than has previously been possible. Also, it is conceivable that the progression of this disabling condition can conceivably be arrested or even reverted.

Currently, there are several problems and unmet needs in the treatment of fibromyalgia, which are addressed by the present invention. All drug treatments available for fibromyalgia target the symptoms but not the mechanisms of the disease. Despite extensive research, the etiology of fibromyalgia was unknown, and thus, there is no disease-modifying therapy available for this condition. By demonstrating that fibromyalgia is causally related to insulin resistance, the present invention teaches methods to modify and treat the disease more specifically and more effectively. Pain in many patients with fibromyalgia is generally decreased but not eliminated with available drugs to treat this disorder (see data in published clinical trials). It is known in the art that available drugs that are routinely used to treat fibromyalgia (FDA approved and off-label drugs commonly used in fibromyalgia) are inadequate to eliminate pain in most patients affected with this disorder. These drugs include tricyclic antidepressants and several norepinephrine reuptake inhibitors (duloxetine, venlafaxine, others) or membrane-stabilizing agents (pregabalin, gabapentin) either alone or in combination. For example, the NNT (numbers needed to treat) for amitriptyline, one of the most effective agents, is 4, followed by duloxetine at 6. This degree of effectiveness of the existing treatments means that at the most, available agents for fibromyalgia work only in some patients and, as seen in published data, only lead to a partial reduction of pain intensity. Most published clinical trials are not only in line with these statements (incomplete pain relief), but also show substantive side effects with the doses needed to achieve clinically meaningful pain relief.

Insulin resistance is a central metabolic abnormality in fibromyalgia, and by addressing it, in conjunction with the use of co-analgesic agents targeting central pain, chronic myofascial pain or fibromyalgia-associated symptoms can be treated more effectively than has previously been possible. As mentioned, the progression of this disabling condition may be arrested or even reverted, particularly with early treatment.

The disclosed formulations and methods are unique and have multiple advantages when compared with other currently existing treatments in that they target the disease mechanism. Additionally, they are more effective in reducing pain. They would allow the medications to be used in lower doses with lesser side effects (a common advantage of drug combinations).

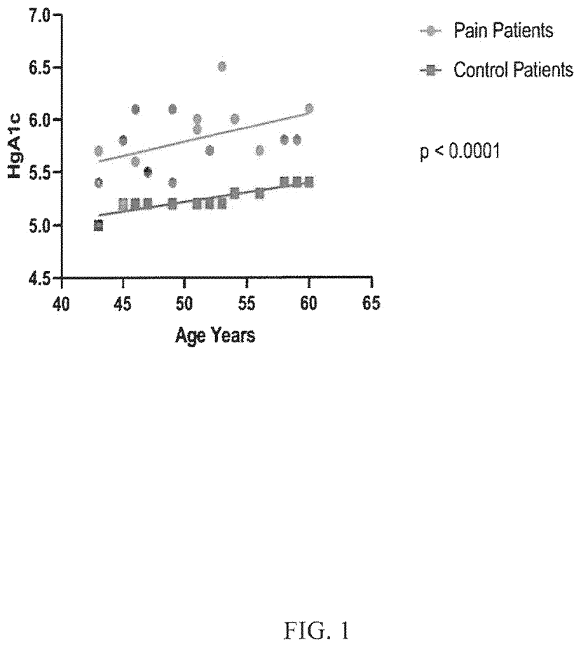

In a first discovery, it is shown that insulin resistance is causatively related to fibromyalgia. For this to be valid, the research findings presented under “the first discovery” and “the second discovery” must be interpreted together. Insulin resistance associates more frequently with some patients with fibromyalgia than in the general population. However, patients with fibromyalgia are subject to physical inactivity due to pain, which leads to weight gain and obesity. Because inactivity and obesity are common causes of insulin resistance, the presence of this abnormality has generally been dismissed or disregarded as an inconsequential secondary association (e.g., unrelated to cause or mechanism of fibromyalgia). In addition, prior studies had demonstrated that insulin resistance is present only in very limited subgroups of fibromyalgia patients. This association was problematic because, for any causative hypothesis to hold water, the putative abnormality (in this case, insulin resistance) needs to be present in most of the subjects affected by the condition (fibromyalgia). A modification of a common diagnostic method was used here to demonstrate that insulin resistance is, indeed, present in most (and perhaps all) patients affected by fibromyalgia, shown in FIG. 1. Specifically, patients with fibromyalgia belong to a distinct population that can be segregated from a normal control group of subjects based on individual HbA1c levels, a surrogate marker of insulin resistance, in an age-stratified manner incorporated into a linear regression model, shown in FIG. 1. The manner in which the data was analyzed was novel and previously not used in any prior studies.

For purposes of this teaching, the patients in this group had met established diagnostic criteria for fibromyalgia, as defined below. Patients (n=23) were selected after a retrospective review of medical records and consisted of consecutive referrals to a subspecialty pain medicine clinic. This retrospective review study was deemed exempt from the requirement of IRB review by an independent ethics review board panel (IntegReview), according to U.S. regulation 45 CFR § 46.104 Category #4. All patients had widespread myofascial pain and had met the 1990 as well as the 2010/2011 American College of Rheumatology criteria for FM diagnosis (i.e., tender points were retained in the evaluation). Patients with comorbid disorders, including a history of cerebrovascular disease, rheumatoid arthritis, untreated endocrine abnormalities, autoimmune conditions or neuromuscular diseases, active malignancy, immunodeficiency, or drug or alcohol abuse, were excluded. Patients taking medications associated with insulin resistance such as glucocorticoids, thiazide diuretics, atypical anti-psychotics, beta-blockers, niacin, statins, and others were also excluded from the sample. The patients underwent laboratory investigations in commercial CLIA (the United States Clinical Laboratory Improvement Act) accredited laboratories that included diagnostic panels for peripheral neuropathy and inflammatory rheumatological disorders, as routinely performed in clinics for most patients with generalized pain disorders. Their laboratory investigations included HbA1c. As controls for the HbA1c results, the values obtained from individuals enrolled in the Framingham Offspring Study (FOS) were used. The results were confirmed by comparison of the fibromyalgia group of patients with a second (different) control population of normal subjects without pain (15) (NHANES; Centers for Disease Control).

From all analytes studied, only the HbA1c levels segregated patients with fibromyalgia from control subjects, as shown in FIG. 1. Despite many patients with fibromyalgia showing HbA1c values within the normal range (<5.7%), a clear-cut difference between the two groups (patients versus controls) came to light when the values in an age continuum were stratified. Virtually all patients with fibromyalgia fell at or above the mean of the control values, as shown in FIG. 1. Statistical analysis between the groups showed highly significant results (p<0.0001) by two statistical tests, i.e., the Wilcoxon ranked pairs signed-rank test (rs Spearman: 0.5556) and by comparison of the linear regression slopes for each group after age stratification of the data. Findings were further confirmed by comparing fibromyalgia patients versus a second control population of normal subjects without pain (NHANES), which yielded virtually identical results (p<0.0001; not shown). Because many patients within the fibromyalgia group were Hispanic, and because Hispanic patients generally have higher HbA1c values, we also compared our population as a group with a third normal Hispanic control group. HbA1c in the fibromyalgia group remained significantly different from the Hispanic control group (not shown).

All prior studies had missed this widespread association because less sensitive methods were used. For example, prior studies had shown an association between fibromyalgia and small fiber neuropathy. Interestingly, insulin resistance is one of the main causes of small fiber neuropathy. However, when the investigators' workup of the causes for the small fiber neuropathy, they used less sensitive markers, and insulin resistance was missed. Another reason for having missed this association is that many patients with fibromyalgia show HbA1c values, which are within the (apparently) normal range (i.e., 5.6 or less). However, these studies demonstrate that the cut-off for normalcy is incorrect for the younger patients, a fact that came to light once the data was subjected to age stratification. Age has a powerful effect on HbA1c levels, which, surprisingly, is generally ignored in most studies involving insulin resistance. Aging leads to a gradual and progressive increase in HbA1c levels; therefore, an HbA1c value of 5.5, for example, does not have the same significance in a 65-year-old person as it does in a 25-year-old person. Based on this concept, the inventor incorporated an age correction into the analysis of the data, and such an approach led to discovery number 1, as illustrated in FIG. 1.

Insulin resistance also has a connection to chronic pain. It shows up in many chronic pain diseases. Chronic pain diseases associated with insulin resistance include but are not limited to idiopathic pain, fibromyalgia, rheumatoid arthritis, degenerative osteoarthrosis, neuropathic pain/neuropathy (large fiber neuropathy, small fiber neuropathy, proximal motor neuropathy, acute mononeuropathies, pressure palsies, radiculopathy); chronic musculoskeletal pain, etc.

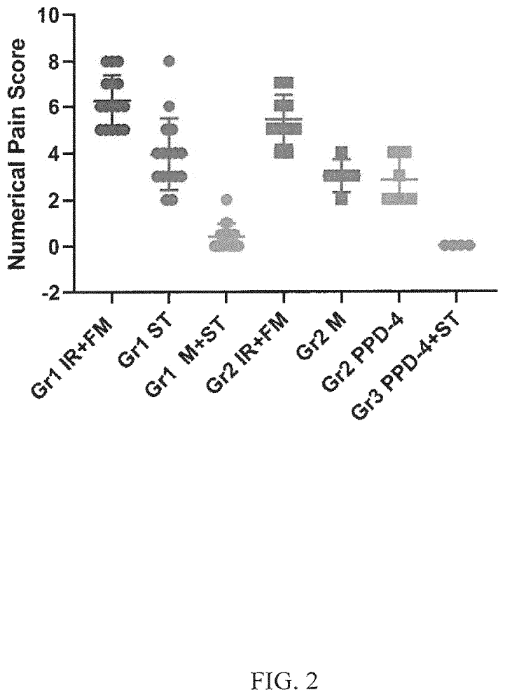

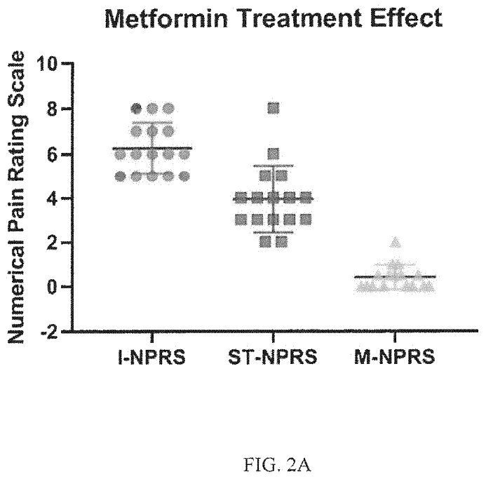

The second discovery should be interpreted in conjunction with the finding described in “discovery 1” to take on meaning. Demonstrating that the association between insulin resistance and fibromyalgia affects most patients with fibromyalgia and not just a few is only half of the solution. An association does not mean causation. The second step towards elucidating the etiology of fibromyalgia relied in a critical piece of data which was missing in the prior art; i.e., demonstrating that treating insulin resistance would lead to an improvement in pain in the affected patients. If insulin resistance were the cause of fibromyalgia, one should improve the disease by treating such abnormality. The pharmacological correction of insulin resistance in these patients resulted in an improvement in the pain scores. These findings, when put together with the data from discovery number 1, confirmed the hypothesis that insulin resistance is probably the cause of fibromyalgia, as shown in FIG. 2. Interestingly, the prior art teaches that metformin may be useful in fibromyalgia, but this piece of information was insufficient. First, metformin was used as an analgesic and a relationship with insulin resistance was never sought by the investigators. Second, prior art teaches that metformin possesses non-specific analgesic effects in chronic pain that are independent of its action on insulin resistance. Therefore, proof was needed that other drugs different from metformin were also effective. To this end, the pain scores of patients that had been diagnosed with insulin resistance and fibromyalgia were reviewed; however, we specifically reviewed the pain scores of patients who were treated with drugs different from metformin (i.e., drugs such as PPD-4 inhibitors and GLP-1 agonists, which work by different mechanisms than metformin). The effect of PPD-4 inhibitors in patients with both fibromyalgia, was equally effective to that of metformin in reducing pain scores, as shown in FIG. 2. This PPD-4 effect confirms that insulin resistance is mechanistically linked fibromyalgia.

The main “discovery,” as it pertains to the treatment for fibromyalgia, is a specific combination of certain drugs that resulted in dramatic effects in pain reduction, not observed with each class of drug alone. All patients receiving a combination of drugs consisting of 1-a drug known to target insulin resistance plus 2-a conventional drug to treat myofascial pain (either amitriptyline, pregabalin, gabapentin, duloxetine and others in these groups), experienced either complete or almost complete resolution of the pain. Such a powerful therapeutic effect was previously unknown in the prior art, as clearly seen in the results of all published clinical trials and data.

In fact, when metformin alone was used, or when other drugs targeting insulin resistance alone (drugs different from metformin, such as PPD-4 inhibitors) were used, there was a reduction in pain scores in most patients with fibromyalgia, but this reduction was only partial, as shown in FIG. 2. Similarly, when drugs previously known to work in fibromyalgia or myofascial pain (such as norepinephrine reuptake inhibitors or membrane-stabilizing agents) were used alone or in combination within their class, again, they do not result in complete elimination of pain in the patients who have fibromyalgia (see data points in Gr1 ST, illustrating this latter point, and also published in several clinical trials). In contrast, by combining drugs in class 1 (such as metformin or PPD-4 inhibitors) with drugs in class 2 (norepinephrine reuptake inhibitors or membrane-stabilizing agents), complete pain resolution was achieved in up to 50% of patients and almost complete resolution in the remainder of the patients afflicted by this disorder as shown in FIG. 2. Such a dramatic degree of improvement was unknown in the prior art.

In summary, based on the data provided in this invention, insulin resistance is causatively related to fibromyalgia. Thus, one can markedly improve pain management in this disorder to extents not previously reported in the prior art.

The drug combinations of the invention can therefore be used not only in patients with fibromyalgia as defined in published criteria, but also in cases of multifocal myofascial pain of unknown etiology which fall short of such criteria (i.e., have a lesser systemic symptom component). Thus, the invention also extends to such patient populations. This is because there are many patients with chronic diffuse myofascial pain indistinguishable from the pain present in patients meeting criteria for fibromyalgia, who do not show readily identifiable causes such specific muscle injuries or myopathies. It is hypothesized that many of the latter patients (i.e., patients with multifocal or diffuse pain which fall shy of meeting criteria for the diagnosis of fibromyalgia) have a similar mechanism driving their chronic pain. Such patients may either represent: 1—early cases of fibromyalgia; 2—patients with chronic pain of central origin which may remain stable as such for their entire lives [i.e., “forme frustre” or incomplete forms of fibromyalgia or incomplete forms of what the National Institutes of Health has defined as “Chronic Overlapping Pain Conditions” (COPC)]. It is important to mention that the latter patients generally exhibit a lesser systemic symptom component and are considered by many experts in the field as part of a fibromyalgia or COPC spectrum disorder. It has been observed in our clinics, that the treatment of such patients having widespread myofascial pain shows similar improvements in pain scores as reported in the population of patients meeting criteria for fibromyalgia. Some experts have also named the condition affecting this latter category of patients (i.e., patients with high fibromyalgia scores but falling short of meeting full diagnostic criteria) as having “Fibromyalgi-ness”.

Prior studies show that metformin alone for the treatment of fibromyalgia leads to a reduction of pain scores. However, metformin alone does not reduce pain to the extents observed with the drug combinations shown in FIG. 2. Here, by combining standard drugs employed conventionally in the treatment of fibromyalgia with drugs used to treat insulin resistance, the analgesic effects of the combination are dramatically enhanced. Interestingly, this effect was maintained when the doses of the drugs used in the standard treatments were decreased (not shown). This latter observation strongly suggests a synergistic effect and opens the exciting prospect of using reduced doses and still achieving excellent clinical outcomes with lesser side effects. Although metformin alone may be partially useful in fibromyalgia, many individuals are intolerant to metformin (at least 20%) or suffer from co-morbid conditions in which metformin is contraindicated. We now teach that other drugs targeting insulin resistance also have analgesic effects in fibromyalgia and that such analgesic effect can be markedly increased in certain drug combinations. Many drugs commonly employed for the treatment of fibromyalgia are associated with weight gain, which in turn worsens insulin resistance (i.e., tricyclic antidepressants, gabapentin, pregabalin). Therefore, while they partially address the symptoms of this disorder, they contribute to the worsening of the disease and accelerating its progression. In contrast, by targeting the disease mechanism we can now potentially decelerate or perhaps halt the progression of this disorder.

Most people with insulin resistance or pre-diabetes and many patients with noninsulin-dependent type 2 diabetes produce enough insulin, but their bodies do not respond to the action of insulin. Also, as people age, their body cells lose some of the ability to respond to insulin. This condition, known as insulin resistance, is associated with an increased incidence of cardiovascular disease and peripheral neuropathy. Insulin resistance is being increasingly recognized to promote or contribute to the development of a broad number of neurological disorders. Fibromyalgia (and probably most patients with “fibromyalgia-ness, central pain or COPC) can now be added to this list of disorders.

The present invention relates to formulations and methods of administering drug combinations (class 1 plus class 2 drugs), to treat pain associated with fibromyalgia and related myofascial pain syndromes. The drug combination can also be useful to treat the associated systemic symptoms (in addition to pain) such as non-restorative sleep, irritable bowel syndrome, fatigue/tiredness, thinking or remembering problem, muscle weakness, headache, pain/cramps in the abdomen, numbness/tingling, dizziness, insomnia, depression, constipation, pain in the upper abdomen, nausea, nervousness, blurred vision, fever, diarrhea, dry mouth, itching, wheezing, Raynaud's phenomenon, hives/welts, ringing in ears, vomiting, heartburn, oral ulcers, loss of/change in taste, seizures, dry eyes, shortness of breath, loss of appetite, rash, sun sensitivity, hearing difficulties, easy bruising, hair loss, frequent urination, painful urination, and bladder spasms. All patients treated in this manner reported marked decreases in the constellation of complaints frequently associated with this disorder.

The drug combinations of the invention can also be useful to treat other chronic pain conditions as mentioned previously and also chronic pain conditions associated with insulin resistance. All patients treated in this manner reported marked decreases in the constellation of complaints frequently associated with this disorder.

Peripheral neuropathies (including small fiber neuropathy) that are associated with insulin resistance may start at very early stages of pre-diabetes. These patients (usually but not always) meet the criteria for pre-diabetes (HbA1c values of 5.7 or higher, up to about 6.4). Patients who are diabetic typically have a HbA1c values of 6.5 or higher. In addition, pursuant to the invention, it is recognized that insulin resistance may be present in patients having an HbA1c value lower than 5.7, particularly in patients who are young (e.g., between the ages of about 20 to about 60 years old). For instance, a 25-year-old individual having an HbA1c value of 5.2 may be experiencing insulin resistance despite showing values of HbA1c below 5.7%. This may also be the case for patients in certain ethnic groups who are particularly susceptible to diabetes (e.g., Hispanic and African Americans). Therefore, the invention contemplates that treatment of fibromyalgia or chronic pain may be accomplished via treating a patient having fibromyalgia or other chronic pain disorder, who may be considered pre-diabetic or to be experiencing insulin resistance at an earlier stage than previously contemplated by HbA1c numbers alone (i.e., patients with HbA1c of <5.6%) with a drug for treating insulin resistance together with a drug for treating fibromyalgia as described herein.

Combination Therapy

Beyond the mentioned therapeutic benefits, there are additional benefits common to many drug combinations. Combination treatments, particularly when packaged as a single pill or tablet for delivery, consisting of two or more active pharmaceutical ingredients, can increase compliance with treatment. Several studies show that a simpler therapy regimen was associated with higher adherence rates. Additionally, combination drugs reduce the administrative costs associated with multiple, separate drugs—such as dispensing costs, insurance co-pays, and separate packaging—as well as the number of prescriptions required for a patient, which in turn delivers substantial cost savings to healthcare systems. It should be emphasized that combination treatments are not available for fibromyalgia.

The disclosed formulations and methods are unique and have multiple advantages when compared with other currently existing treatments in that they target the disease mechanism. Additionally, they are more effective in reducing pain. They would allow the medications to be used in lower doses with lesser side effects (a common advantage of drug combinations).

The present invention is directed to formulations and methods for the treatment of fibromyalgia and related myofascial pain disorders (such as patients with “fibromyalginess” as previously defined). More specifically, the present invention uses a combination of drugs for the treatment of fibromyalgia, related myofascial pain disorders, and associated fibromyalgia symptoms. This combination of drugs consists of one or more drugs in class 1 (see examples below) combined with one or more drugs in class 2 (see examples below). One of the drugs in the combination must always be a drug used to treat insulin resistance. The dose range of each of the drugs used in the combination is outlined below.

Additionally, a small number of patients may benefit from treatment of insulin resistance alone. Therefore, we claim that several drugs that improve insulin resistance (other than metformin) listed in class 1 drugs, alone or in combination with drugs of the same class, can be exclusively used to treat fibromyalgia and related disorders. Because the use of metformin in fibromyalgia was known in the prior art, the use of this drug alone for the treatment of fibromyalgia is not claimed in this application.

Group 1 Drugs

Group (Class) 1 drugs are substances that reduce insulin resistance (IR) or improve glucose intolerance for the treatment of myofascial pain of central origin. Examples of these drugs include biguanides (e.g., metformin), indole-3-propionic acid, PPARγ agonists (natural and synthetic), glucagon-like peptide-1 agonists (GLP-1 agonists), and DPP-4 inhibitors (drugs that inhibit the enzyme which breaks down GLP-1 in mammals). Further examples of biguanide drugs that may be used as a Group 1 drug in the invention include glipizide, glyburide, pioglitazone, repaglinide, saxagliptin, and sitagliptin (in addition to metformin). Examples of GLP-1 agonists that may be used as a Group 1 drug include exenatide, liraglutide, lixisenatide, dulaglutide, and semaglutide. Examples of PPARγ agonists that may be used as a Group 1 drug in the invention include thiazolidinediones (TZDs). More specific examples include rosiglitazone, pioglitazone, troglitazone, flavonoids (such as luteolin, quercetin, kaempferol, (−)-catechin, 2′-hydroxychalone, biochanin A, genistein, 6-hydroxydaiazein, 6′-hydroxy-O-desmethylangolessin), honokiol, amorfrutin 1, amorfrutin B, amorphastilbol, magnolol, resveratrol, polyacetylenes, sesquiterpene lactones (e.g., deoxyelephantopin), diterpene quinone derivatives, and 2-cyano-3,12-dioxo-olean-1,9-dien-28-oic acid (CDDO). Examples of DDP-4 inhibitors that may be used as a Group 1 drug in the invention include sitagliptin, vildagliptin, saxaglipin, and linagliptin.

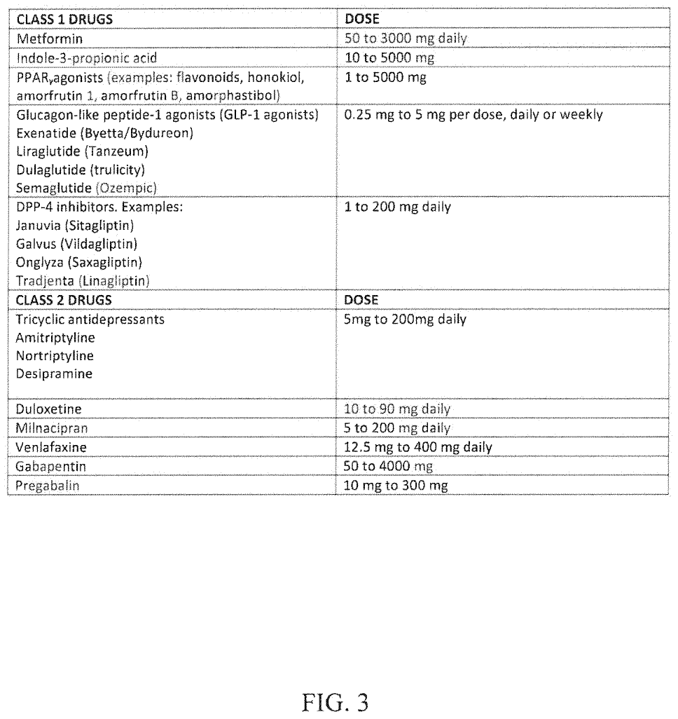

FIG. 3 provides a drug list and dosages for drugs in Group 1. Examples of drugs used to treat insulin resistance include: metformin, glucagon-like peptide-1 agonists, or GLP-1 agonists and PPD-4 inhibitors. The first synthetic drug developed to reduce insulin resistance is exenatide, a synthetic version of exendin-4, a hormone found in the saliva of the Gila monster that was first isolated by John Eng MD in 1992 while working at the Veterans Administration Medical Center in the Bronx, New York. It is a 39-amino-acid peptide, an insulin secretagogue, with glucoregulatory effects. The FDA approved exenatide on Apr. 28, 2005, for patients whose diabetes was not well-controlled on other oral medication. The medication is injected subcutaneously using a filled pen-like device (Byetta), or weekly with either a pen-like device or a conventional syringe (Bydureon). The abdomen is a common injection site after the area is cleaned with an alcohol pad. A new pen must first be tested to see if the medicine is flowing. Commercially, exenatide is produced by direct chemical synthesis. Historically, exenatide was discovered as Exendin-4, a protein naturally secreted in the saliva and concentrated in the tail of the Gila monster.

Exendin-4 shares extensive homology and function with mammalian GLP-1 but has a therapeutic advantage in its resistance to degradation by DPP-IV (which breaks down GLP-1 in mammals) therefore allowing for a longer pharmacological half-life. The biochemical characteristics of Exendin-4 enabled consideration and development of exenatide as a diabetes mellitus treatment strategy. Given this history, exenatide is sometimes referred to as “lizard spit.” Subsequent clinical testing led to the discovery of the also desirable glucagon and appetite-suppressant effects. Glucagon-like peptide-1 receptor agonists, also known as GLP-1 receptor agonists or incretin mimetics, are agonists of the GLP-1 receptor. This class of drugs is used for the treatment of type 2 diabetes. One of their advantages over older insulin secretagogues, such as sulfonylureas or meglitinides, is that they have a lower risk of causing hypoglycemia. There is some concern over the safety profile of these drugs due to proliferative effects in the pancreas. At the same time, diabetes is associated with both acute pancreatitis and pancreatic cancer, and the most recent studies have not found that these drugs can cause either pancreatitis or cancer. Approved GLP-1 agonists include exenatide (Byetta/Bydureon), approved in 2005/2012. Liraglutide (Victoza, Saxenda), approved in 2010, lixisenatide (Lyxumia), approved in 2016 albiglutide (Tanzeum), approved in 2014 by GSK, dulaglutide (Trulicity), approved in 2014 and manufactured by Eli Lilly, Semaglutide (Ozempic).

Dipeptidyl peptidase 4 (DPP-4) inhibitors are another class of drugs used to treat insulin resistance and could be used to treat fibromyalgia pain, DPP-4 inhibitors slow the inactivation and degradation of GLP-1, a hormone involved in glucose removal from the gut. Januvia (Sitagliptin) Galvus (Vildagliptin) Onglyza (Saxagliptin) Tradjenta (Linagliptin) are examples of a growing list of approved drugs for use in the USA known as DPP-4 inhibitors. A DPP-4 inhibitor is preferred in certain embodiments over other Group 1 drugs (particularly Group 1 biguanides such as metformin) because DPP-4 inhibitors are better tolerated than metformin in some patients and are equally effective to metformin as shown in FIG. 2.

Activators of PPARγ may be natural (e.g. flavonoids, honokiol, amorfrutin 1, amorfrutin B, amorphastilbol) or synthetic. They improve metabolic parameters in diabetic animal models and insulin resistance. The natural activators may be better than the synthetic ones because of reduced side effects in comparison to full thiazolidinedione agonists.

The properties and uses of indole-3-propionic acid were initially discovered by the inventor of this application. Higher serum levels of indole propionic acid were found to be associated with a reduced likelihood of diabetes and improved insulin resistance. The putative protective effect of serum indole-propionic acid on the development of diabetes may be explained firstly by its role in modulating incretin secretion from enteroendocrine L cells, more specifically, glucagon-like peptide. Incretin hormones, especially GLP-1, may play a critical role in the pathogenesis of diabetes. Secondly, indole propionic acid has been shown to exert potent anti-oxidative stress capacity suggesting a possible role of this metabolite on protecting β-cell from damage associated with metabolic and oxidative stress, and possibly from amyloid accumulation.

In reference to FIG. 3, dosages of GLP-1 agonists for fibromyalgia treatment range from 0.25 mg to 5 mg per dose. These can be administered daily or weekly. For PPD-4 inhibitors, effective dosages range from 1 to 200 mg daily, depending on the specific inhibitor and patient's therapeutic response for use alone or in combination with other drugs, as described in claim 1 or claim 2. This dose can be administered in one daily dose or several divided doses per day. The dose of metformin, in combination with NSRIs or membrane-stabilizing agents, can range from 50 mg to 3000 mg daily. This dose can be administered in one daily dose or several divided doses per day, either in immediate or extended-release forms. Dosages of antidepressants and norepinephrine reuptake inhibitors (class 2 drugs) to be used in the combination (claim 1) are indicated below. Doses can be administered one time daily or in several divided doses per day, either in immediate or extended-release forms). Tricyclic antidepressants range from 5 mg to 200 mg daily. Duloxetine ranges from 10 to 90 mg daily; venlafaxine from 12.5 mg to 400 mg daily, milnacipran 5 to 200 mg. Dosages for membrane stabilizing agents (class 2 drugs) to be used in the combination are as follows: gabapentin 50 mg to 4000 mg per dose, pregabalin 10 mg to 300 mg per dose. Dosages of PPARγ activators can range from 1 to 5000 mg. Dosages of indole-3-propionic acid can range from 10 to 5000 mg.

In reference to FIG. 1, the association between fibromyalgia and insulin resistance. HbA1c values in 23 patients with FM (8 Hispanic; 11 White; 4 African-American) were compared with the means of a non-diabetic population with normal glucose tolerance (obtained from the Framingham Offspring Study) for the ages stated in the graph. Patients with FM: Green circles (only 18 dots are depicted due to overlapping HbA1c values in some of the patients). Control population (subjects with normal glucose tolerance): Purple squares. Each data point (purple square) in the control population represents the mean of at least 100 subjects.

In reference to FIG. 2, unexpected and dramatic pain improvement with certain drug combinations. Effect of metformin and PPD-4 inhibitors in pain scores, when these drugs were used alone or in combination with other drugs commonly used for the treatment of fibromyalgia (amitriptyline, duloxetine, pregabalin or gabapentin). Numerical pain scores (0-10 scale) are noted in the y-axis. Pain scores are the average of the worse pain experienced in the 7 days prior to the encounter. Gr1 IR+FM: Initial pain scores at the presentation in group 1 which consists of 16 patients with fibromyalgia and insulin resistance. Gr1+ST: Each data point represents the numerical pain score in individual patients from group 1 after receiving standard treatment (pregabalin, gabapentin and/or NSRIs). Note that despite the improvement, this is only partial. Gr1 M+ST: Numerical pain scores in individual patients after the addition of metformin to standard treatment. Note marked improvement, above and beyond that recorded after treatment with each of these classes of drugs alone (ST alone or metformin alone). Gr2 IR+FM: Initial pain scores at the presentation in 10 patients with fibromyalgia and insulin resistance. Gr2 M: Numerical pain scores of patients after receiving metformin alone. Metformin alone led, as previously reported by others, to improvement. However, this improvement was only partial. Gr2 PPD-4: Numerical pain scores after the addition of PPD-4 inhibitors. Improvement in this group was comparable as that noted with metformin alone. Gr3 PPD-4+ST: This group represents 4 patients with insulin resistance and fibromyalgia, who had been treated with both: PPD-4 inhibitors and ST. A review of their pain scores revealed a dramatic improvement. The most powerful and unexpected improvement in pain scores is noted in Gr1 M+ST and in Gr3 PPD4+ST.

In other preferred embodiments, an oral combination of the invention includes sitagliptin (e.g., from about 5 mg/day to about 300 mg/day (and as much as 500 mg/day), preferably from about 100 mg/day to about 150 mg/day) as the therapeutic amount of a drug for treating insulin resistance and chronic pain.

Group 2 Drugs

Group (Class) 2 drugs include substances that have analgesic activity for myofascial pain of central origin, such as certain antidepressants (including those with norepinephrine reuptake inhibition such as duloxetine, milnacipran, venlafaxine, tricyclic antidepressants or others) and/or drugs with membrane-stabilizing properties such as gabapentin and pregabalin. These drug lists are not all-inclusive for all the drugs in this class. Examples of tricyclic antidepressants which may be used as a Group 2 drug in the invention include amitriptyline, desipramine, doxepin, imipramine, nortriptyline, amoxapine, clomipramine, maprotiline, trimipramine, protriptyline, and doxepin. Examples of antidepressants within the class of selective serotonin reuptake inhibitors (SSRIs) which may be used as a Group 2 drug in the invention, include fluoxetine, sertraline, paroxetine, escitalopram, fluvoxamine, citalopram, vilazodone, and vortioxetine. Examples of antidepressants within the class of selective-norepinephrine reuptake inhibitors (SNRIs), which may be used as a Group 2 drug in the invention include venlafaxine, desvenlafaxine, milnacipran, duloxetine, and levo-milnacipran. Examples of atypical antidepressants which may be used as a Group 2 drug in the invention include bupropion, trazodone, and mirtazapine. Examples of useful doses include fluoxetine (initial about 20 mg/day; increasing to up to about 80 mg/day); duloxetine (from about 30 mg/day up to about 60 mg/day); imipramine (from about 50 mg/day to about 150 mg/day); amitriptyline (from about 25 mg/day to about 150 mg/day); nortriptyline (from about 10 mg day to about 160 mg/day); and desipramine (from about 25 mg/day to about 150 mg/day).

FIG. 3 provides a drug list and dosages for drugs in Group 2. In certain preferred embodiments, an oral combination of desipramine (e.g., about 5 mg/day to about 200 mg/day, preferably from about 100 mg/day to about 200 mg/day) is administered in a fixed combination oral dosage form with metformin (e.g., from about 100 mg/day to about 2000 mg/day, preferably from about 250 mg/day to about 1500 mg/day). Desipramine is preferred in certain embodiments over other Group 2 drugs (particularly Group 2 tricyclic antidepressants) because it has less anticholinergic side effects). In other preferred embodiments, a combination of the invention includes venlafaxine (e.g., from about 25 mg/day to about 225 mg/day (and as much as 375 mg/day), preferably from about 75 mg/day to about 150 mg/day) as the therapeutic amount of a drug for treating the pain associated with fibromyalgia. In other preferred embodiments, a DPP-4 inhibitor is preferred over other Group 1 drugs (particularly metformin) when combining it with any of the drugs listed in Group 2.

Administration

The formulations the present invention may be administered by any pharmaceutically effective route. For example, the drugs of Group 1 and/or Group 2 may be formulated in a manner such that they can be administered orally, intranasally, rectally, vaginally, sublingually, buccally, parenterally, or transdermally, and thus, be formulated accordingly. The active drugs in the combination can be administered in liquid, tablet, parenteral, transrectal, transdermal or in any other form of administration suitable in order to achieve a therapeutic effect. Such combination may contain additional fillers, carriers, excipient or excipients, inert or not, known to those skilled in the art of pharmaceutical preparations, in order to provide appropriate volume and/or facilitate absorption of the active drugs in the combination (class 1 plus class 2 drugs).

Different embodiments of the invention include, but are not limited to, the following examples: All possible combinations and permutations of class 1 drugs with class 2 drugs. These include at least one or more drugs that target insulin resistance (class 1 drugs) combined with one or more drugs belonging to class 2. Another embodiment of the invention includes pharmaceutically acceptable complex derivatives of each drug in each group, including solvates, salts, esters, enantiomers, isomers (constitutional and/or stereoisomers), derivatives or prodrugs of each of the drug belonging to either group 1 or group 2. Another embodiment of the invention includes the treatment of associated symptoms of fibromyalgia. Another embodiment of the invention includes using only drugs targeting insulin resistance (i.e., one or more drugs in class 1). Another embodiment of the invention includes multiple variations in the pharmaceutical dosages of each drug in the combination as further outlined below. Another embodiment of the invention includes various forms of preparations including using solids, liquids, immediate or delayed or extended-release forms. Many types of variations are possible as known to those skilled in the art. Another embodiment of the invention includes multiple routes of administration, which may differ in different patients according to their preference, co-morbidities, side effect profile, and other factors (IV, PO, transdermal, etc.). Another embodiment of the invention includes the presence of other substances with the active drugs, known to those skilled in the art, such as fillers, carriers, gels, skin patches, lozenges or other modifications in the preparation to facilitate absorption through various routes (such as gastrointestinal, transdermal, etc.) and/or to extend the effect of the drugs, and/or to attain higher or more stable serum levels or to enhance the therapeutic effect of the active drugs in the combination.

Formulations

In certain embodiments, the drugs of Group 1 and/or Group 2 may be formulated in a pharmaceutically acceptable oral dosage form. Oral dosage forms may include but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent(s) of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the active agent(s) of the present invention formulation. In one embodiment, some or all of the active agent(s) of the present invention particles are coated. In another embodiment, some or all of the active agent(s) of the present invention particles are microencapsulated. In yet another embodiment, some or all of the active agent(s) of the present invention is amorphous material coated and/or microencapsulated with inert excipients. In still another embodiment, the active agent(s) of the present invention particles not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include, but are not limited to, natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g. Avicel, powdered cellulose), and talc. Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include, for example, docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining super disintegrant such as Croscarmellose sodium and different grades of microcrystalline cellulose in different ratios. To aid disintegration, sodium starch glycolate will be added.

In cases where the two (or more) drugs included in the fixed-dose combinations of the present invention are incompatible, cross-contamination can be avoided, e.g.. by incorporation of the drugs in different drug layers in the oral dosage form with the inclusion of a barrier layer(s) between the different drug layers, wherein the barrier layer(s) comprise one or more inert/non-functional materials.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include, but are not limited to, peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as, but not limited to, ethanol, isopropyl alcohol, hexadecyl alcohol, glycerol, and propylene glycol. Ethers, such as but not limited to, poly(ethylene glycol), petroleum hydrocarbons such as mineral oil and petrolatum; and water may also be used in suspension formulations.

In some embodiments, formulations are provided comprising the active agent(s) of the present invention particles described herein and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous the active agent(s) of the present invention particles of consisting of multiple effective particle sizes such that the active agent(s) of the present invention particles having a smaller effective particle size is absorbed more quickly and the active agent(s) of the present invention particles having a larger effective particle size are absorbed more slowly. In certain embodiments, the aqueous dispersion or suspension is an immediate-release formulation. In another embodiment, an aqueous dispersion comprising amorphous the active agent(s) of the present invention particles is formulated such that a portion of the active agent(s) of the present invention particles are absorbed within, e.g., about 3 hours after administration and about 90% of the active agent(s) of the present invention particles are absorbed within, e.g., about 10 hours after administration. In other embodiments, addition of a complexing agent to the aqueous dispersion results in a larger span of the active agent(s) of the present invention containing particles to extend the drug absorption phase such that 50-80% of the particles are absorbed in the first 3 hours and about 90% are absorbed by about 10 hours. Dosage forms for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to the active agent(s) of the present invention particles, the liquid dosage forms may comprise additives, such as (a) disintegrating agents; (b) dispersing agents; (c) wetting agents; (d) at least one preservative, (e) viscosity enhancing agents, (f) at least one sweetening agent, and (g) at least one flavoring agent.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween ® 60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1weight% and propylparaben from about 0.01 to about 0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid the active agent(s) of the present invention formulations can also comprise inert diluents commonly used in the art, such as water or other solvents, solubilizing agents, emulsifiers, and/or sweeteners.

The active agent(s) of the present invention formulations suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agent(s) of the present invention can be dissolved at concentrations of >1 mg/ml using water-soluble beta cyclodextrins (e.g. beta-sulfobutyl-cyclodextrin and 2-hydroxypropylbetacyclodextrin. Proper fluidity can be maintained, for example, by the use of a coating such as a lecithin, by the maintenance of the required particle size in the case of dispersions, and by the use of surfactants. The active agent(s) of the present invention formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The active agent(s) of the present invention suspension formulations designed for extended-release via subcutaneous or intramuscular injection can avoid first-pass metabolism and lower dosages of the active agent(s) of the present invention will be necessary to maintain plasma levels of about 50 ng/ml. In such formulations, the particle size of the active agent(s) of the present invention particles and the range of the particle sizes of the active agent(s) of the present invention particles can be used to control the release of the drug by controlling the rate of dissolution in fat or muscle.

In still other embodiments, effervescent powders containing at least one drug from Group 1 and at least one drug from Group 2 may be prepared. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include e.g: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, a powder comprising the active agent(s) of the present invention formulations described herein may be formulated to comprise one or more pharmaceutical excipients and flavors. Such a powder may be prepared, for example, by mixing the active agent(s) of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

In certain embodiments of the present invention, pharmaceutical compositions containing Group 1 and/or Group 2 drugs may be formulated into a dosage form suitable for parenteral use. For example, the dosage form may be a lyophilized powder, a solution, suspension (e.g., depot suspension).

In other embodiments, pharmaceutical compositions containing Group 1 and/or Group 2 drugs may be formulated into a topical dosage form such as, but not limited to, a patch, a gel, a paste, a cream, an emulsion, liniment, balm, lotion, and ointment.

Tablets of the invention described here can be prepared by methods well known in the art. Various methods for the preparation of the immediate release, modified release, controlled release, and extended-release dosage forms (e.g., as matrix tablets, tablets having one or more modified, controlled, or extended-release layers, etc.) and the vehicles therein are well known in the art. Generally recognized compendium of methods include: Remington: The Science and Practice of Pharmacy, Alfonso R. Gennaro, Editor, 20th Edition, Lippincott Williams & Wilkins, Philadelphia, Pa.; Sheth et al. (1980) Compressed tablets, in Pharmaceutical dosage forms, Vol 1, edited by Lieberman and Lachtman, Dekker, N.Y.

In certain embodiments, solid dosage forms, e.g., tablets, effervescent tablets, and capsules, are prepared by mixing the active agent(s) of the present invention particles with one or more pharmaceutical excipients to form a bulk blend composition. When referring to these bulk blend compositions as homogeneous, it is meant that the active agent(s) of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agent(s) of the present invention formulations can be manufactured by conventional pharmaceutical techniques.

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

Compressed tablets are solid dosage forms prepared by compacting the bulk blend the active agent(s) of the present invention formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the active agent(s) of the present invention formulation. In other embodiments, the film coating aids in patient compliance (e.g., Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, e.g., by placing the bulk blend the active agent(s) of the present invention formulation, described above, inside of a capsule. In some embodiments, the active agent(s) of the present invention formulations (non-aqueous suspensions and solutions) are placed in a soft gelatin capsule. In other embodiments, the active agent(s) of the present invention formulations are placed in standard gelatin capsules or non-gelatin capsules such as capsules comprising HPMC. In other embodiments, the active agent(s) of the present invention formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the active agent(s) of the present invention formulation is delivered in a capsule form. For example, the capsule may comprise between about 100 mg to about 600 mg of the active agent(s) of the present invention. In some embodiments, the capsule may comprise between about 100 to about 500 mg of the active agent(s) of the present invention. In other embodiments, capsule may comprise about 300 mg to about 400 mg of the active agent(s) of the present invention.

In certain preferred embodiments, the formulations of the present invention are fixed-dose combinations of at least one drug from Group 1 and at least one drug from Group 2. Fixed-dose combination formulations may contain the following combinations in the form of single-layer monolithic tablet or multi-layered monolithic tablet or in the form of a core tablet-in-tablet or multi-layered multi-disk tablet or beads inside a capsule or tablets inside a capsule but not limited to: (a) therapeutically efficacious fixed-dose combinations of immediate-release formulations of Group 1 and Group 2 drugs; (b) therapeutically efficacious fixed-dose combinations of immediate release and extended-release Group 1 and Group 2 drugs contained in a single dosage form; (c) therapeutically efficacious fixed-dose combinations of extended-release formulations of Group 1 and Group 2 drugs.

The pharmaceutical compositions described herein can be formulated into any suitable dosage form, including but not limited to, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. In some embodiments, the active agent(s) of the present invention formulations provide a therapeutically effective amount of the active agent(s) of the present invention over an interval of about 30 minutes to about 8 hours after administration, enabling, for example, once-a-day, twice-a-day (b.i.d.), or three times a day (t.i.d.) administration if desired. In one embodiment, the active agent(s) of the present invention particles are formulated into a controlled release or pulsatile solid dosage form for b.i.d. administration. In other embodiments, the active agent(s) of the present invention particles are dispersed in aqueous dispersion for b.i.d. administration. Generally speaking, one will desire to administer an amount of the active agent(s) of the present invention that is effective to achieve a plasma level commensurate with the concentrations found to be effective in vivo for a period of time effective to elicit a desired therapeutic effect.

Depending on the desired release profile, the oral solid dosage forms of the present invention may contain a suitable amount of controlled-release agents, extended-release agents, and/or modified-release agents (e.g., delayed-release agents). The pharmaceutical solid oral dosage forms comprising the active agent(s) of the present invention described herein can be further formulated to provide a modified or controlled release of the active agent(s) of the present invention. In some embodiments, the solid dosage forms described herein can be formulated as a delayed release dosage form such as and enteric-coated delayed release oral dosage forms, i.e., as an oral dosage form of a pharmaceutical composition as described herein which utilizes an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agent(s) of the formulations described herein are delivered using a pulsatile dosage form. Pulsatile dosage forms comprising the active agent(s) of the present invention formulations described herein may be administered using a variety of formulations known in the art. For example, such formulations include, but are not limited to, those described in U.S. Pat. Nos. 5,011,692, 5,017,381, 5,229,135, and 5,840,329, each of which is specifically incorporated by reference. Other dosage forms suitable for use with the active agent(s) of the present invention formulations are described in, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form comprising at least two groups of particles, each containing active agent(s) of the present invention as described herein. The first group of particles provides a substantially immediate dose of the active agent(s) of the present invention upon ingestion by a subject. The first group of particles can be either uncoated or comprise a coating and/or sealant. The second group of particles comprises coated particles, which may comprise from about 2% to about 75%, preferably from about 2.5% to about 70%, or from about 40% to about 70%, by weight of the total dose of the active agent(s) of the present invention in said formulation, in admixture with one or more binders.

Coatings for providing a controlled, delayed, or extended-release may be applied to the drug(s) or to a core containing the drug(s). The coating may comprise a pharmaceutically acceptable ingredient in an amount sufficient, e.g., to provide a delay of from about 2 hours to about 7 hours following ingestion before release of the second dose. Suitable coatings include one or more differentially degradable coatings such as, by way of example only, pH-sensitive coatings (enteric coatings) such as acrylic resins (e.g., Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the active agent(s) of the present invention formulation.

Many other types of controlled/delayed/extended-release systems known to those of ordinary skill in the art and are suitable for use with the active agent(s) of the present invention formulations described herein. Examples of such delivery systems include, e.g., polymer-based systems, such as polylactic and polyglycolic acid, polyanhydrides and polycaprolactone, cellulose derivatives (e.g., ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference. In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric. The controlled/delayed/extended-release systems may utilize a hydrophilic polymer, including but not limited to a water-swellable polymer (e.g., a natural or synthetic gum). The hydrophilic polymer may be any pharmaceutically acceptable polymer which swells and expands in the presence of water to slowly release the active agent(s) of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous-based system and is a spray-dried CAP psuedolatex with particles <1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Extended-release multi-layered matrix tablets may be prepared by using fixed-dose combinations of a drug(s) from Group 1 together with a drug(s) from Group 2. Such formulations may comprise one or more of the drugs within a hydrophilic or hydrophobic polymer matrix. For example, a hydrophilic polymer may comprise guar gum, hydroxypropylmethylcellulose, and xanthan gum as matrix formers. Lubricated formulations may be compressed by a wet granulation method.

Multilayer tablet delivery (e.g., such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (e.g., such as that used in the GeoMatrixTM technology) modifies the hydration/swelling rates of the core and reduces the surface area available for drug release. These partial coatings provide modulation of the drug dissolution profile: they reduce the release rate from the device and shift the typical time-dependent release rate towards constant release. This technology enables customized levels of controlled release of specific drugs and/or simultaneous release of two different drugs at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different drugs or dual release rate of the same drug in a single dosage form may be prepared by using hydrophilic and hydrophobic polymer matrices.

Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In one embodiment, the dosage form is a solid oral dosage form which is an immediate release dosage form whereby >80% of the active agent(s) of the present invention particles hours after administration. In other embodiments, the invention provides an (e.g., solid oral) dosage form that is a controlled release or pulsatile release dosage form. In such instances, the release may be, e.g., 30 to 60% of the active agent(s) of the present invention particles by weight are released from the dosage form within about 2 hours after administration and about 90% by weight of the active agent(s) of the present invention released from the dosage form, e.g., within about 7 hours after administration. In yet other embodiments, the dosage form includes at least one active agent in an immediate-release form and at least one active agent in the delayed-release form, or sustained-release form. In yet other embodiments, the dosage form includes at least two active agents that are released at different rates as determined by in-vitro dissolution testing or via oral administration.

The various release dosage formulations discussed above and others known to those skilled in the art can be characterized by their disintegration profile. A profile is characterized by the test conditions selected. Thus the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tmax, and AUC (0-τ).

In certain embodiments, the controlled, delayed or extended-release of one or more of the drugs of the fixed-dose combinations of the invention may be in the form of a capsule having a shell comprising the material of the rate-limiting membrane, including any of the coating materials previously discussed, and filled with the active agent(s) of the present invention particles. A particular advantage of this configuration is that the capsule may be prepared independently of the active agent(s) of the present invention particles; thus process conditions that would adversely affect the drug can be used to prepare the capsule. Alternatively, the formulation may comprise a capsule having a shell made of a porous or a pH-sensitive polymer made by a thermal forming process. Another alternative is a capsule shell in the form of an asymmetric membrane; i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase- separate by exchanging the solvent with a miscible non-solvent. In another embodiment, spray layered active agent(s) of the present invention particles are filled in a capsule. An exemplary process for manufacturing the spray layered the active agent(s) of the present invention is the fluidized bed spraying process. The active agent(s) of the present invention suspensions or the active agent(s) of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agent(s) of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agent(s) of the present invention particles or the active agent(s) of the present invention complex particles comprise about 30 to 70 wt % of the active agent(s) of the present invention based on the total weight of the particles. In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 100 mg (or at least 300 mg or at least 400 mg) the active agent(s) of the present invention and has a total weight of less than 800 mg (or less than 700 mg). The capsule may contain a plurality of the active agent(s) of the present invention-containing beads, for example, spray layered beads. In some embodiments, the beads are 12-25% the active agent(s) of the present invention by weight. In some embodiments, some or all of the active agent(s) of the present invention containing beads are coated with a coating comprising 6 to 15% (or 8 to 12%) of the total bead weight. Optimization work typically involves lower loading levels and the beads constitute 30 to 60% of the finished bead weight. The capsule may contain a granulated composition, wherein the granulated composition comprises the active agent(s) of the present invention.

The capsule may provide pulsatile release the active agent(s) of the present invention oral dosage form. Such formulations may comprise: (a) a first dosage unit comprising a first the active agent(s) of the present invention dose that is released substantially immediately following oral administration of the dosage form to a patient; (b) a second dosage unit comprising a second the active agent(s) of the present invention dose that is released approximately 3 to 7 hours following administration of the dosage form to a patient. For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In certain embodiments, the formulation may comprise a pulsatile release capsule comprising at least two active agents (e.g., one drug from Group 1 and one drug from Group 2). This pulsatile release capsule may contain a plurality of beads in which some beads are immediate-release beads and other beads are formulated, for example with the use of a coating, for modified release, typically from about 3 to about 10 hours after administration. In other embodiments, the pulsatile release capsule contains a plurality of beads formulated for modified release and the active agent(s) of the present invention powder, for example, spray granulated the active agent(s) of the present invention, for immediate release.

In some embodiments, the release of the active agent(s) of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated and immediate release the active agent(s) of the present invention beads are filled into a soft gelatin capsule. Additional suitable excipients may also be filled with the coated particles in the capsule. The uncoated particles release the active agent(s) of the present invention immediately upon administration while the coated particles do not release the active agent(s) of the present invention until these particles reach the intestine. By controlling the ratios of the coated and uncoated particles, desirable pulsatile release profiles may be obtained. In some embodiments, the ratios between the uncoated and the coated particles are e.g., 20/80, or 30/70, or 40/60, or 50/50, w/w to obtain desirable release.

In certain embodiments, the Group 1 and/or Group 2 drugs contained in a fixed-dose combination of the present invention may be in the form of beads contained within a capsule. In certain embodiments, some beads may release one or both drugs immediately, while other beads would release one or both drugs over an extended period of time or after a delay (delayed-release).

In certain embodiments, spray layered active agent(s) of the present invention particles can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990. In certain embodiments, the spray layered the active agent(s) of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agent(s) of the present invention formulation into the gastrointestinal fluid. In other embodiments, the spray layered the active agent(s) of the present invention particles or spray layered the active agent(s) of the present invention complex particles with enteric coatings described above and one or more excipients are dry blended and compressed into a mass, such as a tablet. In one embodiment, the enteric-coated particles in the tablet substantially avoid the release of the active agent(s) of the present invention, for example, less than 15 wt %, in the stomach but releases substantially all the active agent(s) of the present invention (enterically or sustained-release coated), for example, greater than 80 wt %, in the intestine.

In certain embodiments, a pulsatile release the active agent(s) of the present invention formulation comprises a first dosage unit comprising a formulation made from the active agent(s) of the present invention containing granules made from a spray drying or spray granulated procedure or a formulation made from the active agent(s) of the present invention complex containing granules made from a spray drying or spray granulated procedure without enteric or sustained-release coatings and a second dosage unit comprising spray layered the active agent(s) of the present invention particles or spray layered the active agent(s) of the present invention complex particles with enteric or sustained-release coatings. In one embodiment, the first dosage unit and the second dosage unit are wet or dry blended and compressed into a mass to make a pulsatile release tablet.

In certain embodiments, binding, lubricating and disintegrating agents are blended (wet or dry) to the spray layered the active agent(s) of the present invention to make a compressible blend. The first and second dosage units are compressed separately and then compressed together to form a bilayer tablet. In yet another embodiment, the first dosage unit is in the form of an overcoat and completely covers the second dosage unit.

In certain embodiments, ingredients (including or not including the active agent(s)) of the invention are wet granulated. The individual steps in the wet granulation process of tablet preparation include milling and sieving of the ingredients, dry powder mixing, wet massing, granulation, drying, and final grinding. In various embodiments, the active agent(s) of the present invention composition is added to the other excipients of the pharmaceutical formulation after they have been wet granulated. Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the active agent(s) of the present invention formulation is dry granulated with other excipients in the pharmaceutical formulation. In other embodiments, the active agent(s) of the present invention formulation is added to other excipients of the pharmaceutical formulation after they have been dry granulated.

In other embodiments, the formulation of the present invention formulations described herein is a solid dispersion. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl. 2004/0013734, each of which is specifically incorporated by reference. In some embodiments, the solid dispersions of the invention comprise both amorphous and non-amorphous the active agent(s) of the present invention and can have enhanced bioavailability as compared to conventional the active agent(s) of the present invention formulations. In still other embodiments, the active agent(s) of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval. For example, drug(s) from Group 1 may be administered while the drug(s) from Group 2 is being administered (concurrent administration) or may be administered before or after the drug from Group 2 is administered (sequential administration).

### EXAMPLES

The invention is further illustrated by the following example. Although the invention is explained in relation to a preferred embodiment, it is to be understood that many other possible modifications and variations can be made without departing from the spirit and scope of the invention.

### Example 1

Example 1 is a retrospective chart review of patients with fibromyalgia focusing on potential laboratory abnormalities. When an age correction is applied to the data available for analysis, specifically to the HbA1c values, unexpected findings came to light. Here, it was found that a series of patients with fibromyalgia belong to a distinct population that can be segregated from a control group by the insulin resistance HbA1c values, a biomarker for impaired glucose metabolism, characterized by insulin resistance.

23 patients from a retrospective chart review who were referrals to a subspecialty pain medicine clinic for the treatment of widespread myofascial pain were identified. All patients had met the 1990 as well as the 2010/2011 American College of Rheumatology criteria for fibromyalgia diagnosis (i.e., tender points were retained in the evaluation). Patients with comorbid disorders, including a history of cerebrovascular disease, rheumatoid arthritis, untreated endocrine abnormalities, autoimmune conditions, neuromuscular diseases, active malignancy, immunodeficiency or drug or alcohol abuse were excluded from the sample. Patients taking medications associated with insulin resistance such as glucocorticoids, thiazide diuretics, atypical anti-psychotics, beta-blockers, niacin, statins, and others were also excluded.

The HbA1c values from 23 patients with fibromyalgia (8 Hispanic; 11 White; 4 African-American; 21 females, 2 males) were compared with the HbA1c means of two independent control populations. One was a non-diabetic population with normal glucose tolerance (obtained from the Framingham Offspring Study; FOS NGT) for the ages stated in the graph (FIG. 1). The second population used for confirmation was extracted from the National Health and Nutrition Examination Survey dataset (NHANES non-diabetic) available from the Centers for Disease Control and Prevention (CDC). The data from both control groups can be found published in Pani et. al., Effect of aging on A1C levels in individuals without diabetes: evidence from the Framingham Offspring Study and the National Health and Nutrition Examination Survey 2001-2004. Diabetes Care. 2008; 31(10):1991-6.

Data was obtained from the review of various patient populations as followed: First, the pain scores of two populations of patients were reviewed. Group 1 consisted of 16 patients with fibromyalgia and insulin resistance (HbA1c values of 5.7 or higher) who were prescribed metformin 500 mg twice a day for insulin resistance. In this group, metformin had been added to the “standard treatment” for fibromyalgia. The “standard treatment” (ST) consisted of norepinephrine reuptake inhibitors (amitriptyline, duloxetine or milnacipran) and/or membrane-stabilizing agents (gabapentin or pregabalin). Drugs in the ST group were given to these patients either alone or in combination, depending on patients' preference and/or side effect profile. A retrospective review of numeric pain scores at initial evaluation, after ST and before and after the addition of metformin are reported in the graphic form shown in FIG. 2. The results of these analyses were unexpected in that pain was markedly reduced or eliminated in most patients.

Second, the pain scores of another group of patients with insulin resistance and fibromyalgia, Group 2, who had been treated with PPD-4 inhibitors, a class of drugs that reduce insulin resistance by a mechanism different from metformin were analyzed. In Group 2, 10 patients were identified with fibromyalgia and insulin resistance, 5 of these patients were treated with metformin alone and 5 with a PPD-4 inhibitor alone (not ST had been administered). Their pain scores after treatment with either PPD-4 inhibitors alone or metformin alone showed comparable reductions in pain scores and are shown in FIG. 2 (patients labeled Gr2 MT and Gr2 PPD-4).

Because peripheral neuropathies (including small fiber neuropathy) that are associated with insulin resistance may start at very early stages of pre-diabetes, there is a growing trend among experts to begin early pharmacological interventions to correct this abnormality, particularly when insulin resistance is associated with neuropathy or other risk factors. Following this premise, patients either meeting criteria for pre-diabetes (HbA1c values of 5.7 or higher) or previously undiagnosed diabetes mellitus type 2 were initiated on metformin 500 mg twice a day, added to “standard treatment” for widespread myofascial pain. Standard treatment consisted of either norepinephrine reuptake inhibitors (amitriptyline, duloxetine or milnacipran) and/or membrane-stabilizing agents (gabapentin or pregabalin), depending on tolerability or patients' preference of either class of drug. Pain scores were routinely recorded by the Numeric Pain Rating Scale (NPRS), at each clinical encounter. The NPRS is a unidimensional measure of pain intensity in adults which consists of an 11-point scale for self-reporting of pain. It is one of the most commonly employed instruments in clinical and research settings with extensive evidence supporting its validity (Ferreira-Valente M A, Pais-Ribeiro J L, Jensen M P. Validity of four pain intensity rating scales. Pain. 2011; 152(10):2399-404. (21)). Pain scores were available from the initial evaluation, after ST and after metformin administration.

In order to characterize the variation contributed from the patients, sets of simulated HbA1c data were generated to emulate the populations which were the source of the HbA1c values in the controls. Separately for FOS NGT and NHANES nondiabetic, over the bounded age range from 40 to 69, the tabled values of N, mean, and standard error of each age group were used to estimate the corresponding standard deviation (estimating standard deviation for each age group as the product of the standard error and square root of N). A corresponding simulated source population for the data was generated for each centered age as a set of random normally distributed values with the tabled Ns and means and the calculated standard deviation. Table 1 below provides differences in HbA1c among groups, per Tukey-adjusted differences from regression model:

TABLE 1

| | Estimate | SE | p-value |
|---|---|---|---|
| NHANES Nondiabetic - FOS NGT | 0.20 | 0.02 | <0.0001 |
| Fibromyalgia - FOS NGT | 0.59 | 0.1 | <0.0001 |
| Fibromyalgia - NHANES | 0.39 | 0.1 | 0.0002 |
| Nondiabetic | | | |

Means and standard errors for the resulting simulated data over each age range were verified in good agreement with the values, with estimates of means within 0.1% and standard errors within 4%. This simulated FOS NGT and NHANES non-diabetic HbA1c data, paired with the HbA1c measures from patients with FM, was then modeled by linear regression with relation to age and group (FOS HGT simulated patients (N=1350), NHANES nondiabetic simulated patients (n=1592) versus fibromyalgia patients (n=23). Differences among the groups were estimated by Tukey-adjusted contrasts (Table 2). Differences among pain scores (initial, ST-NPRS, MET-NPRS) were pairwise estimated using the sign test, followed by Hommel adjustment of p-values to compensate for multiple comparisons. Statistical analyses were performed using R statistical software (R Core Team, 2018, version 3.5.1). All statistical tests assumed a 95% level of confidence, with alpha=0.05.

Results

1. Association between fibromalgia and HbAlc levels. From all analytes reviewed, only the HbA1c levels segregated patients with fibromyalgia from control subjects (FIG. 1). Despite many patients with fibromyalgia showing HbA1c values within the normal range (equal or less than 5.6), when the data was stratified in an age continuum and analyzed as described, a clear-cut difference between the groups (patients with fibromyalgia versus controls) came to light. The regression relating HbAlc to group (FOS NGT, NHANES nondiabetic, fibromyalgia HbA1c), summarized in FIG. 1 and Table 1, showed that patients with fibromyalgia average 0.59 units of HbAlc higher than FOS NGT, p<0.0001, and that patients with fibromyalgia average 0.39 units higher than NHANES nondiabetic, p=0.0002.

In FIG. 1, HbA1c values in 23 patients with FM (8 Hispanic; 11 White; 4 African-American; gender: 21 females, 2 males) were compared with the means of two control populations as described in the text. 1—A non-diabetic population with normal glucose tolerance (obtained from the Framingham Offspring Study) and 2—A nondiabetic population from the NHANES data set. Regression lines are shown with shaded 95% confidence regions. FOS NGT and NHANES nondiabetic HbA1c values include scatterplots of published mean values for each age region, while HbA1c results from patients with FM include a scatterplot of measures from individual patients (several overlaps in values). The regression estimates that HbA1c values in patients with FM average 0.59+/−0.1 (mean+/−SE) units higher than FOS NGT (p<0.001), and 0.39 units higher than the NHANES nondiabetic values, p=0.0002.

FIG. 2 depicts the effect of metformin treatment on pain as measured by the NPRS (0-10 scale). GR1 IR+FM means Group 1 patients experiencing insulin resistance and fibromyalgia (16 patients, untreated); GR1 ST means Group 1 patients receiving standard treatment [norepinephrine reuptake inhibitors (amitriptyline, duloxetine or milnacipran)] and/or membrane-stabilizing agents (gabapentin or pregabalin)); Gr1 M+ST means Group patients receiving metformin 500 mg twice daily in addition to standard treatment. Gr2 IR+FM means another group of untreated patients with insulin resistance and fibromyalgia (10 patients); Gr2 M means the Group 2 patients treated with metformin 500 mg twice daily (5 patients) alone; Gr2 PPD-4+ST means Group 2 patients treated with PPD-4 inhibitors (which reduce insulin resistance via a different mechanism than metformin) alone (5 patients); Gr2 PPD-4+ST means Group 2 patients treated with PPD-4 inhibitors and standard treatment (norepinephrine reuptake inhibitors (amitriptyline, duloxetine or milnacipran) and/or membrane-stabilizing agents (gabapentin or pregabalin)).

FIG. 2A is another depiction of the results, in which NPRS for patients who were treated with metformin are reported in graphic form for Group 2 patients I-NPRS: Initial pain scores at presentation; 2-ST-NPRS: Numerical pain scores after standard treatment (pregabalin, gabapentin and/or NSRIs); M-NPRS: Numerical pain scores after addition of metformin. Pain scores are the average of the worse pain experienced in the 7 days prior to the encounter. All pairwise differences among the groups were significant, p<0.0001, per the sign test and following adjustment for multiple comparisons, as shown in Table 2.

Pain scores differed significantly per the sign test among all groups (initial, standard treatment NPRS, metformin plus standard treatment NPRS), with p<0.0001 in each pairwise comparison, as illustrated in FIG. 2A and summarized in Table 2 below:

TABLE 2

| | Median | Min | Q1 | Q3 | Max |
|---|---|---|---|---|---|
| Initial | 8 | 5 | 6.75 | 8 | 8 |
| ST- NPRS | 4 | 2 | 3 | 4.25 | 8 |
| MET - NPRS | 0.25 | 0 | 0 | 0.5 | 2 |

2. Pain scores reduction after treatment of IR. The subgroup of patients who had undergone pharmacological treatment of IR with metformin, in combination with the standard treatment, experienced a dramatic decrease in the pain scores (FIG. 2). Response to metformin plus standard treatment was followed by complete resolution of the pain (report of 0 of 10 in the NPRS) in 8 of 16 patients who had been treated with metformin (50%), a degree of improvement never observed before in such a large proportion of fibromyalgia patients subjected to any available treatment. In contrast, patients treated with standard treatment alone improved, but complete resolution of pain was generally not observed (FIG. 2). Interestingly, some patients responded only to metformin and not to standard treatment with NSRIs or membrane-stabilizing agents. Importantly, there was a long-term retention of the analgesic effect of metformin as noted in Table 3.

The results showed an association between FM and HbA1c levels. Stratification of the values in an age continuum showed a clear-cut difference between the patients and the control groups. As can be visually appreciated in the graph in FIG. 1, almost all patients in the fibromyalgia group fell at or above the mean of the FOS control values, with highly significant differences between the fibromyalgia patients and both control groups (p<0.0001 and p=0.0002 for the FOS and NHANES control populations, respectively). In addition, patients with fibromyalgia in whom insulin resistance had been pharmacologically treated showed dramatic and statistically significant reductions in pain scores (p<0.0001 for all groups) as shown in FIG. 2 and Table 2.

### CONCLUSION

Example 1 shows that most (if not all) patients with fibromyalgia belong to a distinct population that can be segregated from a control group by the insulin resistance glycated hemoglobin A1c (HbA1c) levels, a surrogate marker of insulin resistance (insulin resistance). This was demonstrated by analyzing the data after introducing an age stratification correction into a linear regression model. This strategy showed highly significant differences between fibromyalgia patients and control subjects (p<0.0001 and p=0.0002, for two separate control populations, respectively). A subgroup of patients meeting criteria for pre-diabetes or diabetes (patients with HbA1c values of 5.7% or greater) who had undergone treatment with metformin showed dramatic improvements of the insulin resistance widespread myofascial pain, as shown by the insulin resistance scores using a pre- and post-treatment numerical pain rating scale (NPRS) for evaluation.

The drawings and specific descriptions of the drawings are intended to be read in conjunction with the entirety of these disclosures. The formulations and methods for treatment of fibromyalgia and related myofascial pain disorders may, however, be embodied in many different forms and should not be construed as being limited to the embodiments set forth in the preceding claim; rather, these embodiments are provided by way of illustration only and so that this disclosure will be thorough and convey understanding to those skilled in the art.

### CITATIONS

1. Wolfe F, Ross K, Anderson J, Russell L T, Hebert L. The prevalence and characteristics of fibromyalgia in the general population. Arthritis Rheum. 1995; 38(1):19-28.

2. van Eijk-Hustings Y, Kroese M, Creemers A, Landewe R, Boonen A. Resource utilisation and direct costs in patients with recently diagnosed fibromyalgia who are offered one of three different interventions in a randomised pragmatic trial. Clin Rheumatol. 2016; 35(5):1307-15.

3. Boomershine C S. Fibromyalgia: the prototypical central sensitivity syndrome. Curr Rheumatol Rev. 2015; 11(2):131-45.

4. Ablin J N, Buskila D. Update on the genetics of the fibromyalgia syndrome. Best Pract Res Clin Rheumatol. 2015; 29(1):20-8.

5. Ablin J N, Bar-Shira A, Yaron M, Orr-Urtreger A. Candidate-gene approach in fibromyalgia syndrome: association analysis of the genes encoding substance P receptor, dopamine transporter and alphal-antitrypsin. Clin Exp Rheumatol. 2009; 27(5 Suppl 56):533-8.

6. Staud R. Fibromyalgia pain: do we know the source? Curr Opin Rheumatol. 2004; 16(2):157-63.

7. Staud R. Cytokine and immune system abnormalities in fibromyalgia and other central sensitivity syndromes. Curr Rheumatol Rev. 2015; 11(2):109-15.

8. Dell'Osso L, Bazzichi L, Baroni S, Falaschi V, Conversano C, Carmassi C, et al. The inflammatory hypothesis of mood spectrum broadened to fibromyalgia and chronic fatigue syndrome. Clin Exp Rheumatol. 2015; 33(1 Suppl 88):5109-16.

9. Frosch O H, Yau P L, Osorio R S, Rusinek H, Storey P, Convit A. Insulin resistance among obese middle-aged is associated with decreased cerebrovascular reactivity. Neurology. 2017; 89(3):249-55.

10. Mountz J M, Bradley L A, Modell J G, Alexander R W, Triana-Alexander M, Aaron L A, et al. Fibromyalgia in women. Abnormalities of regional cerebral blood flow in the thalamus and the caudate nucleus are associated with low pain threshold levels. Arthritis Rheum. 1995; 38(7):926-38.

11. Grembiale R D, Ursini F, Bruno C, Savarino F, Naty S. AB1098 Obesity and insulin resistance in fibromyalgia patients. Annals of the Rheumatic Diseases. 2013; 71(Suppl 3):700-.

12. Fava A, Plastino M, Cristiano D, Spano A, Cristofaro S, Opipari C, et al. Insulin resistance possible risk factor for cognitive impairment in fibromialgic patients. Metab Brain Dis. 2013; 28(4):619-27.

13. Wolfe F, Smythe H A, Yunus M B, Bennett R M, Bombardier C, Goldenberg D L, et al. The American College of Rheumatology 1990 Criteria for the Classification of Fibromyalgia. Report of the Multicenter Criteria Committee. Arthritis Rheum. 1990; 33(2):160-72.

14. Wolfe F, Clauw D J, Fitzcharles M A, Goldenberg D L, Hauser W, Katz R S, et al. Fibromyalgia criteria and severity scales for clinical and epidemiological studies: a modification of the ACR Preliminary Diagnostic Criteria for Fibromyalgia. J Rheumatol. 2011; 38(6):1113-22.

15. Pani L N, Korenda L, Meigs J B, Driver C, Chamany S, Fox C S, et al. Effect of aging on A1C levels in individuals without diabetes: evidence from the Framingham Offspring Study and the National Health and Nutrition Examination Survey 2001-2004. Diabetes Care. 2008; 31(10):1991-6.

16. Oaklander A L, Herzog Z D, Downs H M, Klein M M. Objective evidence that small-fiber polyneuropathy underlies some illnesses currently labeled as fibromyalgia. Pain. 2013; 154(11):2310-6.

17. Kosmidis M L, Koutsogeorgopoulou L, Alexopoulos H, Mamali I, Vlachoyiannopoulos P G, Voulgarelis M, et al. Reduction of Intraepidermal Nerve Fiber Density (IENFD) in the skin biopsies of patients with fibromyalgia: a controlled study. Journal of the neurological sciences. 2014; 347(1-2):143-7.

18. Alcocer-Gomez E, Garrido-Maraver J, Bullon P, Marin-Aguilar F, Cotan D, Carrion A M, et al. Metformin and caloric restriction induce an AMPK-dependent restoration of mitochondrial dysfunction in fibroblasts from Fibromyalgia patients. Biochim Biophys Acta. 2015; 1852(7):1257-67.

19. Bullon P, Alcocer-Gomez E, Carrion A M, Marin-Aguilar F, Garrido-Maraver J, Roman-Malo L, et al. AMPK Phosphorylation Modulates Pain by Activation of NLRP3 Inflammasome. Antioxid Redox Signal. 2016; 24(3):157-70.

20. Lee Y H, Song G G. Comparative efficacy and tolerability of duloxetine, pregabalin, and milnacipran for the treatment of fibromyalgia: a Bayesian network meta-analysis of randomized controlled trials. Rheumatol Int. 2016; 36(5):663-72.

21. Gilron I, Chaparro L E, Tu D, Holden R R, Milev R, Towheed T, et al. Combination of pregabalin with duloxetine for fibromyalgia: a randomized controlled trial. Pain. 2016; 157(7):1532-40.

22. Staud R, Price D D, Robinson M E. The provisional diagnostic criteria for fibromyalgia: one step forward, two steps back: comment on the article by Wolfe et al. Arthritis Care Res (Hoboken). 2010; 62(11):1675-6; author reply 6-8.

23. Wolfe F, Clauw D J, Fitzcharles M A, Goldenberg D L, Hauser W, Katz R L, et al. 2016 Revisions to the 2010/2011 fibromyalgia diagnostic criteria. Semin Arthritis Rheum. 2016; 46(3):319-29.

24. Wolfe F, Clauw D J, Fitzcharles M A, Goldenberg D L, Katz R S, Mease P, et al. The American College of Rheumatology preliminary diagnostic criteria for fibromyalgia and measurement of symptom severity. Arthritis Care Res (Hoboken). 2010; 62(5):600-10.

25. Talbot K, Wang H Y, Kazi H, Han L Y, Bakshi K P, Stucky A, et al. Demonstrated brain insulin resistance in Alzheimer's disease patients is associated with IGF-1 resistance, IRS-1 dysregulation, and cognitive decline. J Clin Invest. 2012; 122(4):1316-38.

26. Watson K, Nasca C, Aasly L, McEwen B, Rasgon N. Insulin resistance, an unmasked culprit in depressive disorders: Promises for interventions. Neuropharmacology. 2018; 136(Pt B):327-34.

27. Athauda D, Foltynie T. Insulin resistance and Parkinson's disease: A new target for disease modification? Prog Neurobiol. 2016; 145-146:98-120.

28. Chyan Y J, Poeggeler B, Omar R A, Chain D G, Frangione B, Ghiso J, et al. Potent neuroprotective properties against the Alzheimer beta-amyloid by an endogenous melatonin-related indole structure, indole-3-propionic acid. J Biol Chem. 1999; 274(31):21937-42.

29. de Mello V D, Paananen J, Lindstrom J, Lankinen M A, Shi L, Kuusisto J, et al. Indolepropionic acid and novel lipid metabolites are associated with a lower risk of type 2 diabetes in the Finnish Diabetes Prevention Study. Sci Rep. 2017; 7:46337.

What is claimed is:

1. A pharmaceutical composition comprising a therapeutically effective amount of a drug that treats insulin resistance, and a therapeutic amount of a drug for treating the pain associated with fibromyalgia.

2. The pharmaceutical composition of claim 1, wherein the drug for treating insulin resistance is selected from the group consisting of a biguanide, indole-3-propionic acid, a PPARγ agonist, a glucagon-like peptide-1 (GLP-1) agonist, a DPP-4 inhibitor, and combinations of any of the foregoing.

3. The pharmaceutical composition of claim 1, wherein the drug for treating the pain associated with fibromyalgia is selected from the group consisting of a tricyclic antidepressant, a selective serotonin reuptake inhibitor, a selective norepinephrine reuptake inhibitor, an atypical antidepressant, a drug with membrane-stabilizing properties, and combinations of any of the foregoing.

4. The pharmaceutical composition of claim 2, wherein the drug which treats insulin resistance is a biguanide selected from the group consisting of glipizide, glyburide, pioglitazone, repaglinide, saxagliptin, sitagliptin, and metformin.

5. The pharmaceutical composition of claim 2, wherein the drug which treats insulin resistance is a GLP-1 agonist selected from the group consisting of exenatide, liraglutide, lixisenatide, dulaglutide, and semaglutide.

6. The pharmaceutical composition of claim 2, wherein the drug which treats insulin resistance is a PPARγ agonist.

7. The pharmaceutical composition of claim 2, wherein the drug which treats insulin resistance is a DDP-4 inhibitor selected from the group consisting of sitagliptin, vildagliptin, saxagliptin, and linagliptin.

8. The pharmaceutical composition of claim 3, wherein the drug for treating pain associated with fibromyalgia is a tricyclic antidepressant selected from the group consisting of amitriptyline, desipramine, doxepin, imipramine, nortriptyline, amoxapine, clomipramine, maprotiline, trimipramine, and protriptyline.

9. The pharmaceutical composition of claim 3, wherein the drug for treating the pain associated with fibromyalgia is an atypical antidepressant selected from the group consisting of bupropion, trazodone, and mirtazapine.

10. The pharmaceutical composition of claim 3, wherein the drug for treating the pain associated with fibromyalgia is a selective norepinephrine reuptake inhibitor (SNRI) selected from the group consisting of venlafaxine, desvenlafaxine, milnacipran, duloxetine, and levomilnacipran.

11. The pharmaceutical composition of claim 3, wherein the drug for treating pain associated with fibromyalgia is a selective serotonin reuptake inhibitor (SSRI) selected from the group consisting of fluoxetine, sertraline, paroxetine, escitalopram, fluvoxamine, citalopram, vilazodone, and vortioxetine.

12. The pharmaceutical composition of claim 3, wherein the drug for treating the pain associated with fibromyalgia is a drug with membrane-stabilizing properties selected from the group consisting of gabapentin, pregabalin, carbamazepine and oxcarbazepine.

13. The pharmaceutical composition of claim 1, wherein the therapeutic amount of a drug for treating insulin resistance is from about 100 mg to about 2000 mg metformin.

14. The pharmaceutical composition of claim 1, wherein the therapeutic amount of a drug for treating the pain associated with fibromyalgia comprises from about 25 mg to about 200 mg desipramine.

15. The pharmaceutical composition of claim 1, wherein the therapeutic amount of a drug for treating the pain associated with fibromyalgia comprises from about 10 mg to about 375 mg venlafaxine.

16. The pharmaceutical composition of claim 1, wherein the therapeutically effective amount of a drug that treats insulin resistance comprises from about 1 mg to about 500 mg sitagliptin.

17. A method for treating fibromyalgia in humans, comprising chronically administering to a human patient experiencing fibromyalgia who is experiencing insulin resistance a therapeutically effective amount of a drug that treats insulin resistance and a therapeutically effective amount of a drug for treating the pain associated with fibromyalgia.

18. The method of claim 17, wherein the drug for treating insulin resistance is selected from the group consisting of a biguanide, indole-3-propionic acid, a PPARγ agonist, a glucagon-like peptide-1 (GLP-1) agonist, a DPP-4 inhibitor, and combinations of any of the foregoing and the drug for treating pain associated with fibromyalgia is selected from the group consisting of a tricyclic antidepressant, a selective serotonin reuptake inhibitor, a selective norepinephrine reuptake inhibitor, an atypical antidepressant, a drug with membrane stabilizing properties, and combinations of any of the foregoing.

19. A method for treating chronic pain in humans, comprising chronically administering to a human patient experiencing chronic pain who is experiencing insulin resistance a therapeutically effective amount of a drug that treats insulin resistance and a therapeutically effective amount of a drug for treating the pain associated with fibromyalgia.

20. The method of claim 19, wherein the drug for treating insulin resistance is selected from the group consisting of a biguanide, indole-3-propionic acid, a PPARγ agonist, a glucagon-like peptide-1 (GLP-1) agonist, a DPP-4 inhibitor, and combinations of any of the foregoing and the drug for treating pain associated with fibromyalgia is selected from the group consisting of a tricyclic antidepressant, a selective serotonin reuptake inhibitor, a selective norepinephrine reuptake inhibitor, an atypical antidepressant, a drug with membrane-stabilizing properties, and combinations of any of the foregoing.
